(19) United States
(12) Patent Application Publication
(10) Pub. No.: US 2016/0256480 A1
(43) Pub. Date: Sep. 8, 2016

(54) THERAPEUTIC COMPOSITION FOR WOUND HEALING

(71) Applicant: Wintley PHIPPS, Vero Beach, FL (US)

(72) Inventor: Wintley PHIPPS, Vero Beach, FL (US)

(21) Appl. No.: 15/060,042

(22) Filed: Mar. 3, 2016

Related U.S. Application Data
(60) Provisional application No. 62/127,667, filed on Mar. 3, 2015.

(51) Int. Cl. A61K 31/716; A61K 45/06

(52) U.S. Cl. CPC A61K 31/716; A61K 45/06

(57) ABSTRACT

A therapeutic composition comprising a beta-glucan, at east one fatty acid, and at least one secondary polysaccharide, which may increase a rate of wound healing. A method for healing a wound in a subject, by administering to a subject in need thereof, the therapeutic composition as described.

### CROSS-REFERENCE TO RELATED APPLICATIONS

This application claims priority to provisional application 62/127,667, filed Mar. 3, 2015, the entire contents of which are incorporated herein by reference.

### BACKGROUND

1. Technical Field

The present disclosure relates to a composition of beta-glucans, a triglyceride, and a saccharide to aid in wound healing.

2. Description of the Related Art

The “background” description provided herein is for the purpose of generally presenting the context of the disclosure. Work of the presently named inventor, to the extent it is described in this background section, as well as aspects of the description which may not otherwise qualify as prior art at the time of filing, are neither expressly or impliedly admitted as prior art against the present disclosure.

Glucans are important secondary metabolites isolated from plants and micro-organisms. Glucans are generally described as a polysaccharide of D-glucose monomers, linked by glycosidic bonds. They exhibit prophylactic and therapeutic properties, and can function as biological response modifiers when administered to mammals. As such, glucans have shown beneficial effects in the treatment of infectious and autoimmune diseases, and in clinical management of cancer.

Glucans target various cell types in the immune system, and more particularly macrophages. Previous research has shown that glucans display protective properties against experimentally induced infections in mammalian model systems. Specifically, glucans exert their function on macrophages, monocytes, lymphocytes, and other immune cells in the mammalian system that plays a significant role in elicitation of the immune response.

For example, administration of glucans has been shown to significantly enhance the immune system in animals to a wide variety of experimentally induced bacterial, viral, fungal and parasitic infections. Glucans also show strong anti-tumor activity. Glucan carries out its biological function by binding to specific receptor molecules located on the surface of macrophages. In in vitro studies, exposure of these cells to beta-glucans stimulates the immune system. One representative glucan with such immune-enhancing characteristics is branched beta(1,3)-glucan (referred hereinafter to as beta-glucan or β-glucan).

Beta-glucans are naturally occurring polymers of saccharides (polysaccharides) and are constituents of the cell wall of certain pathogenic bacteria, algae, fungi, and cereals (e.g. oats, barley, rye, wheat). The healing and immunostimulating properties of mushrooms have been known for thousands of years in the Eastern countries. These mushrooms contain biologically active polysaccharides that mostly belong to group of beta-glucans. Beta-glucans can increase host immune defense by enhancing macrophages and natural killer cell function. The induction of cellular responses by beta-glucans is likely to involve their specific interaction with several cell surface receptors, such as complement receptor 3 (CR3; CD11b/CD18), lactosylceramide, selected scavenger receptors, and dectin-1 (betaGR). As an immunostimulating agent, which acts through the activation of macrophages and NK cell cytotoxicity, beta-glucan can inhibit tumor growth in promotion stage also. The interaction between glucan and its receptor produce further stimulatory effects such as enhanced phagocytosis, increased cell size, enhanced cell proliferation, enhanced adherence and chemotactic activity and production of a wide range of cytokines and leukotrienes.

Cytokines are critical to a myriad of fundamental homeostatic and pathophysiological processes such as fever, wound healing, inflammation, tissue repair and fibrosis. They play important roles in regulating cell function such as proliferation, migration, and matrix synthesis. It is the balance or the net effect of the complex interplay between these mediators, which appears to play a major role in regulating the initiation, progression and resolution of wounds. Wound healing involves a complex process including induction of acute inflammation by the initial injury, followed by parenchymal and mesenchymal cell proliferation, migration, and activation with production and deposition of extracellular matrix.

These clinical effects are highly beneficial and important and represent an opportunity to develop novel pharmaceutics based on beta-glucans derived from a variety of sources, such pharmaceutics having potentially wide application in both veterinary and human medicine.

### BRIEF SUMMARY OF THE DISCLOSURE

A significant impediment to the effective use of beta-glucans as an immune-modulating agent is the rate at which they are absorbed in mammals when orally administered. Naturally occurring beta-glucans contain cross-linked polymers of the basic glucose units and are considered very large molecules. They are insoluble in water and are acid resistant. Thus, when administered orally, they pass through the stomach virtually intact due to their large molecular weight which hinders optimal absorption in the gastrointestinal tract.

Absorbability of these large-molecule beta-glucans is further reduced by the lack of specific enzymes in the gastrointestinal tract to break down large molecular weight, polymeric beta-glucans. It is generally thought that large molecular weight, water-insoluble glucan preparations are unlikely to efficiently bind to its specific receptors and be internalized in the cell at high enough concentrations to be effective as immune-modulators.

The lower gastrointestinal tract offers an alternative route of administration of beta-glucans. Rectal administration uses the rectum as a route of administration for medication and other fluids, which are absorbed through passive diffusion into the rectum's blood vessels and flow into the body's circulatory system which distributes the drug to the body's organs and bodily systems. Additionally, wounds in the rectum as well as the genital region are often difficult to treat, requiring surgical attention, extensive pain in recovery and difficulty for a patient to return to normal activity. Thus it may be highly beneficial to patients to develop beta glucans for ailments of the rectum and genital regions.

In view of the forgoing, an objective of the present disclosure is to present a therapeutic composition that may improve wound healing noninvasively.

According to a first aspect, a therapeutic composition including a beta-glucan, at least one fatty acid selected from the group consisting of arachidic acid, linoleic acid, linolenic acid, myristic acid, oleic acid, palmitic acid, palmitoleic acid, and stearic acid, and at least one secondary polysaccharide, wherein the therapeutic composition increases a rate of wound healing.

In some formulations, the therapeutic composition may further include glycerinated gelatin.

In some formulations, the therapeutic composition may further include synthetic fatty acids of a carbon chain length of 6 carbons to 22 carbons.

In some formulations of the therapeutic composition, the beta-glucan is curdlan, laminaran, pachymaran, lentinan, pleuran, schizophyllan, sclerotinan, sclero-beta-glucan, grifolan, yeast beta-glucan, or barley beta-glucan.

In some formulations, the therapeutic composition may further include a triglyceride, wherein the triglyceride comprises at least one fatty acid selected from the group consisting of arachidic acid, linoleic acid, linolenic acid, myristic acid, oleic acid, palmitic acid, palmitoleic acid, and stearic acid.

In some formulations of the therapeutic composition, the beta-glucan is derived from Euglena gracilis, bacteria, a mushroom fungus, a yeast, and/or at least one cereal source selected from the group consisting of oat, barley, wheat, and rye.

In some formulations of the therapeutic composition, the beta-glucan is synthetically prepared.

In some formulations of the therapeutic composition, the beta-glucan is a linear polymer and/or a branched polymer.

In some formulations of the therapeutic composition, the secondary polysaccharide is at least one polysaccharide selected from the group consisting of arrabinogalactan, fucoidan, pectin, and galactomannan.

In some formulations of the therapeutic composition, the beta-glucan is sulfonated, phosphorylated, aminated, and/or nitrated.

In some formulations of the therapeutic composition, the rate of wound healing increases by at least 1.25 times to 25 times relative to the rate of wound healing without the therapeutic composition.

In some formulations of the therapeutic composition, the beta-glucan comprises about 0.1% to about 50% by weight, relative to the total weight of the therapeutic composition.

In some formulations of the therapeutic composition, the fatty acid comprises about 0.1% to about 45% by weight, relative to the total weight of the therapeutic composition.

In some formulations of the therapeutic composition, the secondary polysaccharide comprises about 0.1% to about 20% by weight, relative to the total weight of the therapeutic composition.

In some formulations of the therapeutic composition, the therapeutic composition is in a form of a tablet, capsule, suspension, cream, ointment, lotion, powder, gel, solution, paste, spray, foam, oil, enema, suppository, a slow release matrix.

According to a second aspect, a method for healing a wound in a subject, which includes administering to a subject in need thereof a therapeutic composition, which includes a beta-glucan derived from Euglena gracilis, bacteria, a mushroom fungus, a yeast, and/or at least one cereal source selected from the group consisting of oat, barley, wheat, and rye, at least one fatty acid selected from the group consisting of arachidic acid, linoleic acid, linolenic acid, myristic acid, oleic acid, palmitic acid, palmitoleic acid, and stearic acid, and at least one secondary polysaccharide selected from the group consisting of arabinogalactan, fucoidan, pectin, galactomannan. The therapeutic composition increases a rate of wound healing by at least 1.25 times to 25 times relative to the rate of wound healing without the therapeutic composition.

In some implementations of the method, the therapeutic composition further includes a glycerinated gelatin, and/or synthetic fatty acids, comprising a carbon chain length of 6 carbons to 22 carbons, and/or a triglyceride, comprising at least one fatty acids selected from the group consisting of arachidic acid, linoleic acid, linolenic acid, myristic acid, oleic acid, palmitic acid, palmitoleic acid, and stearic acid.

In some implementations of the method, the therapeutic composition is administered in a cavity of the subject.

In some implementations of the method, the therapeutic composition is administered in a form of a capsule, a suspension, a cream, an ointment, a lotion, a powder, gel, a solution, a paste, a spray, a foam, an oil, an enema, a suppository, or a slow release matrix.

In some implementations of the method, the wound is an anal fissure or a vaginal tear.

The foregoing paragraphs have been provided by way of general introduction, and are not intended to limit the scope of the following claims. The described formulations and implementations, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is a test of mouse macrophages with 3 mg/ml of beta-glucan incubation at 200×, 0 hours;

FIG. 1B is a test of mouse macrophages with 3 mg/ml of beta-glucan incubation at 400×, 0 hours;

FIG. 1C is a test of mouse macrophages with 3 mg/m of beta-glucan incubation at 200×, 20 hours;

FIG. 1D is a test of mouse macrophages with 3 mg/m of beta-glucan incubation at 400×, 20 hours;

FIG. 1E is a test of mouse macrophages with 3 mg/ml of beta-glucan incubation at 200×, 48 hours;

FIG. 1F is a test of mouse macrophages with 3 mg/ml of beta-glucan incubation at 400×, 48 hours;

FIG. 2A is a test of mouse macrophages with 1 mg/ml of beta-glucan incubation at 200×, 0 hours;

FIG. 2B is a test of mouse macrophages with 1 mg/ml of beta-glucan incubation at 400×, 0 hours;

FIG. 2C is a test of mouse macrophages with 1 mg/ml of beta-glucan incubation at 200×, 20 hours;

FIG. 2D is a test of mouse macrophages with 1 mg/ml of beta-glucan incubation at 400×, 20 hours;

FIG. 2E is a test of mouse macrophages with 1 mg/ml of beta-glucan incubation at 200×, 48 hours;

FIG. 2F is a test of mouse macrophages with 1 mg/ml of beta-glucan incubation at 400×, 48 hours;

FIG. 3A is a test of mouse macrophages with 200 μg/ml of beta-glucan incubation at 200×, 0 hours;

FIG. 3B is a test of mouse macrophages with 200 μg/ml of beta-glucan incubation at 400×, 0 hours;

FIG. 3C is a test of mouse macrophages with 200 μg/ml of beta-glucan incubation at 200×, 20 hours;

FIG. 3D is a test of mouse macrophages with 200 μg/ml of beta-glucan incubation at 400×, 20 hours;

FIG. 3E is a test of mouse macrophages with 200 μg/ml of beta-glucan incubation at 200×, 48 hours;

FIG. 3F is a test of mouse macrophages with 200 μg/ml of beta-glucan incubation at 400×, 48 hours;

FIG. 4A is a test of mouse macrophages with 50 μg/ml of beta-glucan incubation at 200×, 0 hours;

FIG. 4B is a test of mouse macrophages with 50 μg/ml of beta-glucan incubation at 400×, 0 hours;

FIG. 4C is a test of mouse macrophages with 50 μg/ml of beta-glucan incubation at 200×, 20 hours;

FIG. 4D is a test of mouse macrophages with 50 μg/ml of beta-glucan incubation at 400×, 20 hours;

FIG. 4E is a test of mouse macrophages with 50 μg/ml of beta-glucan incubation at 200×, 48 hours;

FIG. 4F is a test of mouse macrophages with 50 μg/ml of beta-glucan incubation at 400×, 48 hours;

### DETAILED DESCRIPTION OF THE EMBODIMENTS

Throughout the specification ranges may he expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The term “wound” as used herein refers to any break in the epithelium resulting from a cut, tear, abrasion, adhesion, surgical incision, thermal, chemical, or friction burn, ulcer, or the like, as a result of an accident, incident, surgical procedure, or the like. Wound can be further defined as acute and/or chronic.

Referring now to the drawings, wherein like reference numerals designate identical or corresponding parts throughout the several views.

The present disclosure relates to a therapeutic composition including a beta-glucan, at least one fatty acid, and at least one secondary polysaccharide. The therapeutic composition may increases a rate of wound healing. In some formulations of the therapeutic composition, the rate of wound healing increases by at least 1.25 times to 25 times relative to the rate of wound healing without the therapeutic composition.

The beta-glucans may be derived from a variety of natural sources including, but not limited to Euglena, a protist, bacteria, such as gram-positive or gram negative bacteria including Escherichia, Streptococcus, Salmonella, Serratia, Shigella, Pseudomonas, Neisseria, Haemophilus, Agrobacterium, or Rhizobium; a mushroom fungus, such as Chinese Reishi (Ganoderma lucidum), or Japanese Shitake (Lentinula edodes) and Maitake (Grifola frondosa), arboreal fungi: Chaga (Inonotus obliquus), Turkey Tail (Trametes versicolor), Split Gill (Schizophyllum commune), Mulberry Yellow Polypore (Phellinus linteus) and cultivated fungi, Hiratake (Pleurotus ostreatus, Oyster mushroom); a yeast, such as Schizosaccharomyces pombe, Saccharomyces cerevisiae, or Saccharomyces pastorianus; and/or a cereal source, such as oat, barley, wheat, rye, sorghum, or rice.

The beta-glucans included in the therapeutic composition may include, curdlan, laminaran, pachymaran, lentinan, pleuran, schizophyllan, sclerotinan, sclero-beta-glucan, grifolan, yeast beta-glucan, or barley beta-glucan. Beta-glucans derived from different sources vary in molecular weight. The beta-glucans included in the therapeutic composition may have a molecular weight about 10 kDa to about 500 kDa, about 20 kDa to about 450 kDa, about 50 kDa to about 400 kDa, about 75 kDa to about 350 kDa, about 100 kDa to about 300 kDa, about 125 kDa to about 275 kDa, about 150 kDa to about 250 kDa, or about 175 kDa to about 225 kDa.

In some formulations of the therapeutic composition, the beta-glucan is synthetically prepared. Beta-glucans may be synthetically prepared by methods including solid phase synthesis or solution phase synthesis. The methods may include beta-glucan derived from the natural sources as described herein, and synthetically combined with beta-glucans prepared by solid phase or solution phase synthesis methods. The beta-glucan may obtained from commercial products such as Drago-Beta Glucan, SymGlucan®, or the like.

Beta-glucans may be linear or branched to varying degrees. Beta-glucans incorporated in some formulations of the therapeutic composition may be about 0.1% to about 99% branched, about 0.5% to about 90% branched, about 1% to about 85% branched, about 1.5% to about 80% branched, about 2% to about 70% branched, about 2.5% to about 6% branched, about 3% to about 50% branched, about 3.5% to about 40% branched, about 4% to about 30% branched, about 4,5% to about 20% branched, about 5% to about 18% branched, about 6% to about 16% branched, about 7% to about 14% branched, about 8% to about 12% branched, about 9% to about 10% branched, relative to the total beta-glucan content.

Some beta-glucans are water insoluble, but in some formulations of the therapeutic composition, the beta-glucan is sulfonated, phosphorylated, aminated, and/or nitration. For example, beta-glucans may be sulfonated by chlorosulfonic acid addition to a solution of beta-glucan. For example, phosphorylation of beta-glucan may be achieved by mechanochemical phosphorylation methods employing a planetary ball mill. For example, amination of beta-glucan may be accomplished by oxidizing an hydroxyl of a terminal glucose of the beta-glucan to an aldehyde by an oxidizing agent such as sodium periodate, then adding sodium triacetoxyborohydride to reduce the aldehyde to an amine. For example, nitration of beta-glucans may be achieved by adding nitric acid and sulfuric acid to a solution of beta-glucan. Beta-glucan may be best solubilized in a polar aprotic solvent such as dimethyl sulfoxide, acetonitrile, or other polar-aprotic solvents known in the art.

As described herein the therapeutic composition includes at least one of the following fatty acids, arachidic acid, linoleic acid, linolenic acid, myristic acid, oleic acid, palmitic acid, palmitoleic acid, and stearic acid. A fatty acid is a carboxylic acid with a long aliphatic chain, which is either saturated or unsaturated. Most naturally occurring fatty acids have an unbranched chain of an even number of carbon atoms, from 4 to 28. The fatty acids that may be incorporated into the therapeutic composition may be derived from or replaced by cocoa butter, shea butter, coconut oil, palm oil, rapeseed oil, soybean oil, cottonseed oil or a combination thereof. The fatty acids employed in the present therapeutic composition may allow for improved penetration of the beta-glucans into a tissue contacting the therapeutic composition. The fatty acids may allow for more rapid induction of wound healing and reduce a dosage of the therapeutic composition.

In some formulations, the therapeutic composition may further include synthetic fatty acids of a carbon chain length of 6 carbons to 22 carbons. Synthetic fatty acids may produce a similar effect of rapid induction of wound healing, but may also provide for improved solubility of the beta glucan. For example, the synthetic fatty acids may include, but are not limited to 2-hydroxyoleic acid, 2-hydroxyarachidonic acid, and 2-hydroxydocosahexaenoic acid. In some formulations of the therapeutic composition, the synthetic fatty acid comprises about 0.1% to about 10% by weight, about 0.5% to about 9.5% by weight, about 1% to about 9% by weight, about 2% to about 8% by weight, about 3% to about 7% by weight, or about 4% to about 6% by weight, relative to the total weight of the therapeutic composition.

In some formulations, the therapeutic composition further includes a triglyceride. The triglyceride is an ester derived from glycerol and three fatty acids. The triglyceride that may be included in the therapeutic composition comprises at least one fatty acid as described above. The triglyceride included in the present therapeutic composition may further include triglycerides of caproic acid, caprilic acid, capric acid, lauric acid, behenic acid and the like. The triglyceride may be included as a pure form or in a mixture of many combinations of triglycerides. In some formulations of the therapeutic composition, the triglyceride comprises about 0.1% to about 45% by weight, about 1% to about 40% by weight, about 5% to about 35% by weight, about 10% to about 30% by weight, about 12% to about 28% by weight, about 15% to about 25% by weight, or about 18% to about 22% by weight, relative to the total weight of the therapeutic composition.

The therapeutic composition further includes at least one secondary polysaccharide. In some formulations of the therapeutic composition, the secondary polysaccharide may include, but is not limited to arabinogalactan, fucoidan, pectin, laminarin, porphyrin, and galactomannan. The secondary polysaccharide may be defined as polymeric carbohydrate molecules composed of long chains of monosaccharide units bound together by glycosidic linkages and on hydrolysis give the constituent monosaccharides or oligosaccharides. The secondary polysaccharide may be derived from bacterial, fungal, yeast, or algal cell walls, or may be synthetically prepared. The secondary polysaccharide may enhance induction of wound healing by recruiting macrophages (i.e. white blood cells and the like) that detect the secondary polysaccharides in the tissue contacted by the therapeutic composition.

In some formulations, the therapeutic composition may further include glycerinated gelatin. Glycerinated gelatin is a jellylike preparation that is made from glycerin, gelatin, and water and is commonly used as a base for suppositories, ointments, and medicaments familiar to those in the art. Glycerinated gelatin may improve solubility of the beta-glucan, fatty acids, polysaccharides, and triglycerides that may be included in the therapeutic composition. In some formulations of the therapeutic composition, the glycerinated gelatin comprises about 0.1% to about 20% by weight, about 0.5% to about 18% by weight, about 1% to about 15% by weight, about 5% to about 12% by weight, or about 8% to about 10% by weight, relative to the total weight of the therapeutic composition.

In some formulations of the therapeutic composition, the beta-glucan comprises about 0.1% to about 50% by weight, about 0.5% to about 45% by weight, about 1% to about 40% by weight, about 5% to about 35% by weight, about 10% to about 30% by weight, about 12% to about 28% by weight about 15% to about 25% by weight, or about 18% to about 22% by weight, relative to the total weight of the therapeutic composition.

In some formulations of the therapeutic composition, the fatty acid comprises about 0.1% to about 45% by weight, about 1% to about 40% by weight, about 5% to about 35% by weight, about 10% to about 30% by weight, about 12% to about 28% by weight, about 15% to about 25% by weight, or about 18% to about 22% by weight, relative to the total weight of the therapeutic composition.

In some formulations of the therapeutic composition, the secondary polysaccharide comprises about 0.1% to about 20% by weight, about 1% to about 18% by weight, about 2% to about 16% by weight, or about 5% to about 12% by weight, about 7% to about 10% by weight, relative to the total weight of the therapeutic composition.

In some formulations of the therapeutic composition, the therapeutic composition is in a form of a tablet, capsule, suspension, cream, ointment, lotion, powder, gel, solution, paste, spray, foam, oil, enema, suppository, a slow release matrix. In order to prepare the composition in any of the forms above, several components may be included in trace amounts such as polyethylene glycol (PEG), various alcohols, and/or monosaccharides, disaccharides, and oligosaccharides.

In some formulations, PEGs that may be included in the therapeutic composition may have an average molecular weight of about 200-20,000, about 500-15,00, about 1,000-10,000, about 1,200-9,000, about 1,500-8,000, about 2,000-7,000, about 3,000-6,000, about 4,000-5,000.

In some formulations, the various alcohols which may be included in the therapeutic composition may include, but are not limited to ethyl alcohol, butyl alcohol, octyl alcohol, lauryl alcohol, myristyl alcohol, cetyl alcohol, stearyl alcohol, oleyl alcohol, isostearyl alcohol, dimeric alcohols, ethylene glycol, propylene glycol, butylene glycol, hexylene glycol, dodecane diol, hexadecane diol, octadecane diol, and 1,3,5-pentane triol.

In some formulations, the monosaccharides, disaccharides, or oligosaccharides which may be included in the therapeutic composition may include, but are not limited to erythritol, pentaerythritol, diglycerine, triglycerine, dipentaerythritol, sorbitol, sorbitan, sorbide, glucose, fructose, mannose, xylose, methyl glucoside, trehalose, sucrose, ethylenediamine and the like.

In some formulations, the therapeutic composition may further include water, propylene glycol, glycerine, propylene glycol fatty acid ester, sucrose fatty acid ester, sorbitan fatty acid ester, polyoxethylene alkyl ether, polyoxyethylene polyhydric alcohol fatty acid ester and oil-soluble polyoxyalkylene glycol derivative in trace amounts less than about 2% by weight, less than 1.75% by weight, less than 1.5% by weight, less than 1.25% by weight, less than 1% by weight, less than 0.75% by weight, less than 0.5% by weight, less than 0.25% by weigh, or less than 0.1% by weight, relative to the total weight of the therapeutic composition.

In some formulations of the therapeutic composition PEG, fatty acids, triglycerides, water, propylene glycol, glycerine, propylene glycol fatty acid ester, sucrose fatty acid ester, sorbitan fatty acid ester, polyoxethylene alkyl ether, polyoxyethylene polyhydric alcohol fatty acid ester and oil-soluble polyoxyalkylene glycol derivative may be included in order to lower a viscosity of the therapeutic composition, to adjust a melting point, to improve a lubricating property, or to promote the penetration of the beta-glucan.

The present disclosure further relates to a method for healing a wound in a subject, which includes administering to a subject in need thereof the therapeutic composition as described herein. The therapeutic composition may increase a rate of wound healing, relative to the rate of wound healing without the therapeutic composition, by at least 1.25 times to 25 times, by at least 1.5 times to 22 times, by at least 1.75 times to 20 times, by at least 2 times to 18 times, by at least 2.5 times to 16 times, by at least 3 times to 15 times, by at least 3.5 times to 14 times, by at least 4 times to 13 times, by at least 5 times to 10 times, by at least 6 times to 8 times.

In some implementations of the method, the therapeutic composition is administered in a form of a capsule, a suspension, a cream, an ointment, a lotion, a powder, gel, a solution, a paste, a spray, a foam, an oil, an enema, a suppository, or a slow release matrix.

In some implementations of the method, the therapeutic composition is administered in a cavity of the subject. The cavity may include a urethra, a vaginal cavity, a rectal cavity, an anal cavity, or a sinus cavity. Two examples of wounds that commonly occur in the aforementioned cavities are an anal fissure or a vaginal tear. These particular ailments are often difficult to treat and located in sensitive areas of the anatomy.

The examples below are intended to further illustrate beta-glucan in a therapeutic composition and are not intended to limit the scope of the claims.

### EXAMPLE 1

### Method

J774A.1 cells are mouse monocyte/macrophage cells. These cells are adherent and upon death will detach from the surface fo the culture flasks or coverslips. Cells were grown to approximately 80% confluence in 24-well plates on 12 mm coverslips overnight. Cells were grown in a minimum essential medium with insulin (IMEMZO, Irvine Scientific, Santa Ana, Calif.) with 5% fetal bovine serum (FBS, Atlanta Biologicals, Norcross, Ga.). Beta-glucan was dissolved with MilliQ Ultrapure water. The beta-glucan was only partially dissolved. Beta-glucan was diluted with IMEMZO media and added to the cells for the duration of the incubation. Cells were examined daily and phase-contrast images taken at specific intervals to capture the health of the cells. All images were captured at 200× magnification.

The beta-glucan was purchased from Sigma-Aldrich (CAS Number 9051-97-2; MDL number MFCD00466918). Beta-glucan source was Euglena gracilis. The beta-1,3-Glucan from Euglena gracilis is comparable to beta-glucan from yeast, bacteria, and mushroom fungus sources.

### RESULTS

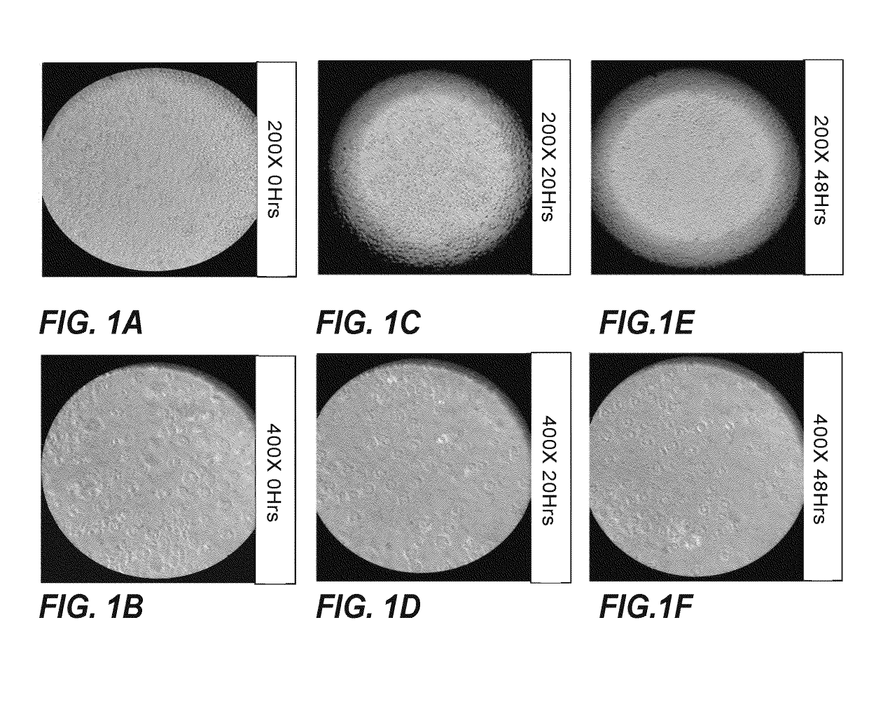

FIG. 1A-FIG. 1F depicts the highest level of beta-glucan incubated with the mouse macrophages, 3 mg/ml. At 3 mg/ml, beta-glucan did not pose any visible pathological effects on cellular growth or survival. Cells did not decrease in size and remained adherent over the 48 hour culture period. Cells increased in numbers at a rate that is typical in the base media of IMEMZO. Images were taken under either 200× magnification phase contrast (FIG. 1A, 1C, 1E) or 400× phase contrast (FIG. 1B, 1D, 1F)

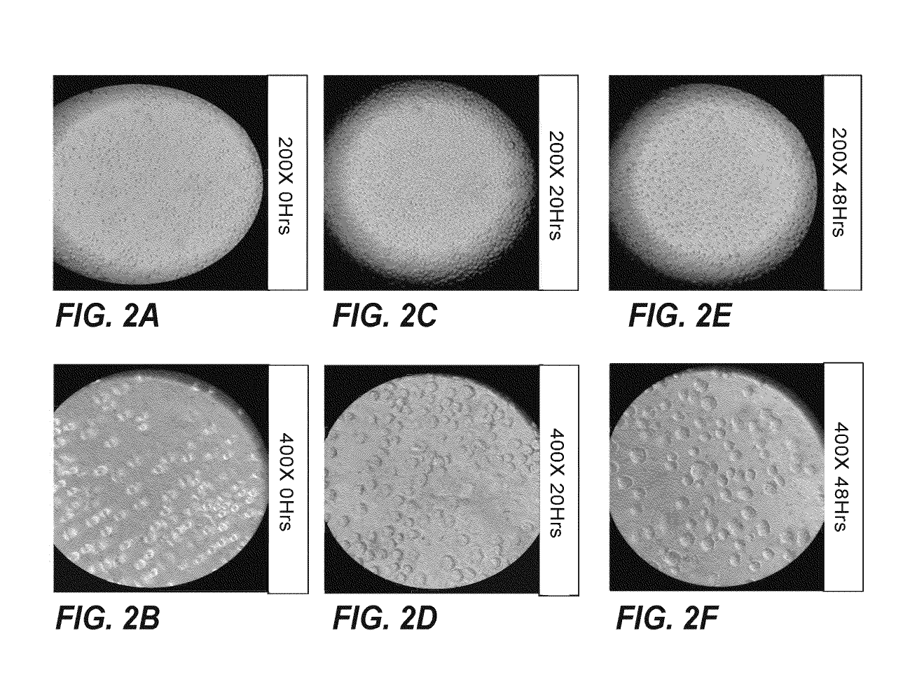

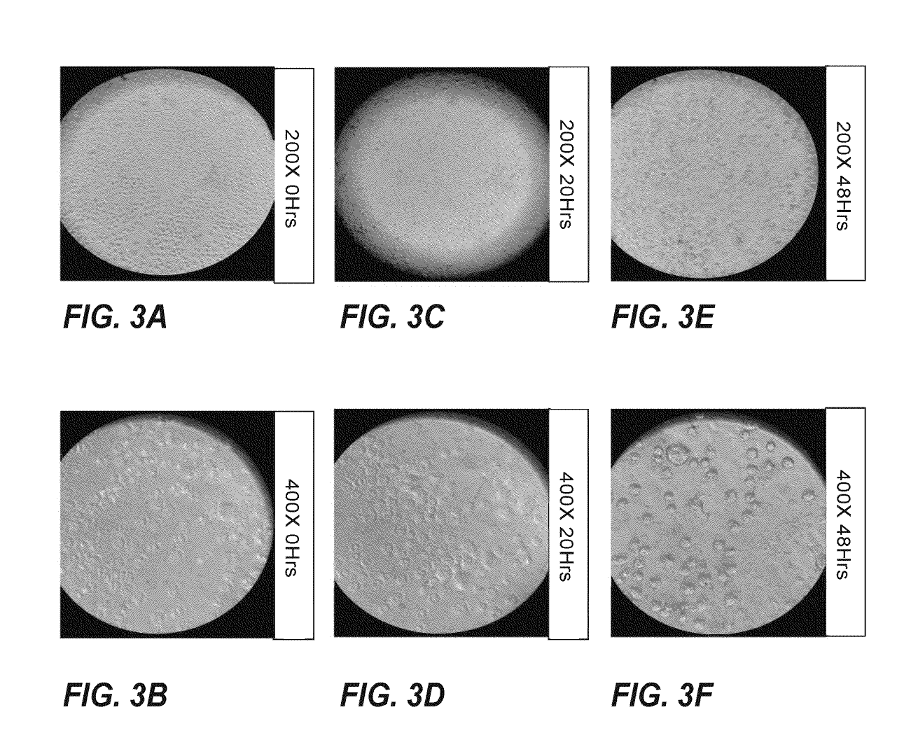

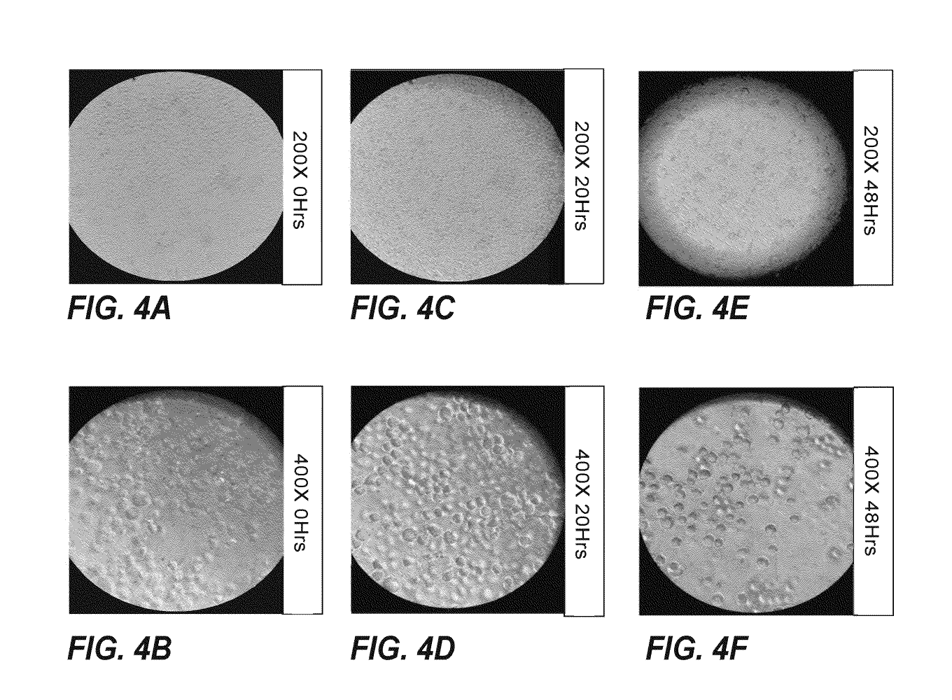

FIG. 2 depicts mouse macrophages incubated with 1 mg/ml of beta-glucan over 48 hours, with no adverse affect on the cell growth and proliferation. Similar results are shown in FIG. 3 with 200 μg/ml of beta-glucan over 48 hours, and in FIG. 4, with 50 μg/ml of beta-glucan over 48 hours. Indicating that mouse macrophage are not adversely affected by the beta-glucan. Images were taken under either 200× magnification phase contrast (FIG. 2A, 2C, 2E; FIG. 3A, 3C, 3E; FIG. 4A, 4C, 4E) or 400× phase contrast (FIG. 2B, 2D, 2F; FIG. 3B, 3D, 3F; FIG. 4B, 4D, 4F).

1: A therapeutic composition comprising:
a beta-glucan;
at least one fatty acid selected from the group consisting of arachidic acid, linoleic acid, linolenic acid, myristic acid, oleic acid, palmitic acid, palmitoleic acid, and stearic acid; and
at least one secondary polysaccharide;
wherein the therapeutic composition increases a rate of wound healing.

2: The therapeutic composition of claim 1, further comprising glycerinated gelatin.

3: The therapeutic composition of claim 1, further comprising synthetic fatty acids of a carbon chain length of 6 carbons to 22 carbons.

4: The therapeutic composition of claim 1, wherein the beta-glucan is curdlan, laminaran, pachymaran, lentinan, pleuran, schizophyllan, sclerotinan, sclero-beta-glucan, grifolan, yeast beta-glucan, or barley beta-glucan.

5: The therapeutic composition of claim 1, further comprising a triglyceride, wherein the triglyceride comprises at least one fatty acid selected from the group consisting of arachidic acid, linoleic acid, linolenic acid, myristic acid, oleic acid, palmitic acid, palmitoleic acid, and stearic acid.

6: The therapeutic composition of claim 1, wherein the beta-glucan is derived from Euglena gracilis, bacteria, a mushroom fungus, a yeast, and/or at least one cereal source selected from the group consisting of oat, barley, wheat, and rye.

7: The therapeutic composition of claim 1, wherein the beta-glucan is synthetically prepared.

8: The therapeutic composition of claim 1, wherein the beta-glucan is a linear polymer and/or a branched polymer.

9: The therapeutic composition of claim 1, wherein the at least one secondary polysaccharide is selected from the group consisting of arabinogalactan, fucoidan, pectin, and galactomannan.

10: The therapeutic composition of claim 1, wherein the beta-glucan is sulfonated, phosphorylated, aminated, and/or nitrated.

11: The therapeutic composition of claim 1, wherein the rate of wound healing increases by at least 1.25 times to 25 times relative to the rate of wound healing without the therapeutic composition.

12: The therapeutic composition of claim 1, wherein the beta-glucan comprises about 0.1% to about 50% by weight, relative to the total weight of the therapeutic composition.

13: The therapeutic composition of claim 1, wherein the fatty acid comprises about 0.1% to about 45% by weight, relative to the total weight of the therapeutic composition.

14: The therapeutic composition of claim 1, wherein the secondary polysaccharide comprises about 0.1% to about 20% by weight, relative to the total weight of the therapeutic composition.

15: The therapeutic composition of claim 1, wherein the therapeutic composition is in a form of a tablet, capsule, suspension, cream, ointment, lotion, powder, gel, solution, paste, spray, foam, oil, enema, suppository, a slow release matrix.

16: A method for healing a wound in a subject which comprises administering to a subject in need thereof:
a therapeutic composition which comprises:
a beta-glucan derived from Euglena gracilis, bacteria, a mushroom fungus, a yeast, and/or at least one cereal source selected from the group consisting of oat, barley, wheat, and rye.
at least one fatty acid selected from the group consisting of arachidic acid, linoleic acid, linolenic acid, myristic acid, oleic acid, palmitic acid, palmitoleic acid, and stearic acid; and
at least one secondary polysaccharide selected from the group consisting of arabinogalactan, fucoidan, pectin, galactomannan;
wherein the therapeutic composition increases a rate of wound healing by at least 1.25 times to 25 times relative to the rate of wound healing without the therapeutic composition.

17: The method of claim 16, further comprising glycerinated gelatin, and/or synthetic fatty acids, comprising a carbon chain length of 6 carbons to 22 carbons, and/or a triglyceride, comprising at least one fatty acids selected from the group consisting of arachidic acid, linoleic acid, linolenic acid, myristic acid, oleic acid, palmitic acid, palmitoleic acid, and stearic acid.

18: The method of claim 16, wherein the therapeutic composition is administered in a cavity of the subject.

19: The method of claim 16, wherein the therapeutic composition is administered in a form of a capsule, a suspension, a cream, an ointment, a lotion, a powder, gel, a solution, a paste, a spray, a foam, an oil, an enema, a suppository, or a slow release matrix.

20: The method of claim 16, wherein the wound is an anal fissure or a vaginal tear.
